(19) United States
(12) Patent Application Publication
(10) Pub. No.: US 2007/0106374 A1
(43) Pub. Date: May 10, 2007

(54) Radiopaque coating for biomedical devices

(75) Inventors: David A. Glocker, West Henrietta, NY (US); Mark M. Romach, Spencerport, NY (US)

Correspondence Address: SIMPSON & SIMPSON, PLLC, 5555 MAIN STREET, WILLIAMSVILLE, NY, 14221-5406, US

(73) Assignee: Isoflux, Inc., Rochester, NY (US)

(21) Appl. No.: 11/586,836

(22) Filed: Oct. 26, 2006

Related U.S. Application Data
(63) Continuation-in-part of application No. 11/151,583, filed on Jun. 13, 2005.
(63) Continuation-in-part of application No. 11/087,909, filed on Mar. 23, 2005.
(63) Continuation-in-part of application No. 11/040,433, filed on Jan. 21, 2005.
(60) Provisional application No. 60/579,577, filed on Jun. 14, 2004.
(60) Provisional application No. 60/555,721, filed on Mar. 23, 2004.
(60) Provisional application No. 60/538,749, filed on Jan. 22, 2004.

(51) Int. Cl. A61F 2/02; A61F 2/82; C23C 14/32

(52) U.S. Cl. 623/1.34; 623/1.46; 600/431; 427/2.24; 204/192.1

(57) ABSTRACT

A medical device has a porous metallic coating that can withstand the high strains inherent in the use of such devices without delamination. A coating of the metal is applied to a medical device, such as a stent, by vapor deposition so that the thermomechanical properties of the stent are not adversely affected. The coating preferable has high emissivity. The coating is applied via a generally oblique coating flux or a low energy coating flux.

### CROSS REFERENCE TO RELATED APPLICATIONS

This application is:
a continuation-in-part of U.S. patent application Ser. No. 11/151,583 filed Jun. 13, 2005 that in turn claims the benefit of U.S. provisional application No. 60/579,577 filed Jun. 14, 2004, and
a continuation-in-part of U.S. patent application Ser. No. 11/087,909 filed Mar. 23, 2005 that claims the benefit of U.S. provisional application No. 60/555,721 filed Mar. 23, 2004 and is a continuation-in-part of U.S. patent application Ser. No. 11/040,433 filed Jan. 21, 2005 that claims the benefit of U.S. provisional application No 60/538,749 filed Jan. 22, 2004;
the entire disclosures of which are incorporated herein by reference in their entirety for any and all purposes.

### TECHNICAL FIELD

The present invention relates to medical devices.

### BACKGROUND

Stents have become extremely important devices in the treatment of cardiovascular disease. A stent is a small mesh “scaffold” that can be positioned in an artery to hold it open, thereby maintaining adequate blood flow. Typically a stent is introduced into the patient's system through the brachial or femoral arteries and moved into position using a catheter and guide wire. This minimally invasive procedure replaces surgery and is now used widely because of the significant advantages it offers for patient care and cost.

In order to deploy a stent, it must be collapsed to a fraction of its normal diameter so that it can be manipulated into the desired location. Therefore, many stents and guide wires are made of an alloy of nickel and titanium, known as nitinol, which has the unusual properties of superelasticity and shape memory. Both of these properties result from the fact that nitinol exists in a martensitic phase below a first transition temperature, known as M_{f}, and an austenitic phase above a second transition temperature, known as A_{f}. Both M_{f} and A_{f} can be manipulated through the ratio of nickel to titanium in the alloy as well as thermal processing of the material. In the martensitic phase nitinol is very ductile and easily deformed, while in the austenitic phase it has a high elastic modulus. Applied stresses produce some martensitic material at temperatures above A_{f} and when the stresses are removed the material returns to its original shape. This results in a very springy behavior for nitinol, referred to as superelasticity or pseudoelasticity. Furthermore, if the temperature is lowered below M_{f} and the nitinol is deformed, when the temperature is raised above A_{f} it will recover its original shape. This is described as shape memory.

Stents having superelasticity and shape memory can be compressed to small diameters, moved into position, and deployed so that they recover their full size. By choosing an alloy composition having an A_{f} below normal body temperature, the stent will remain expanded with significant force once in place. Remarkably, during this procedure the stent, e.g. of nitinol, must typically withstand strain deformations of as much as 8%.

Stents and similar intraluminal devices can also be made of materials like stainless steel and other metal alloys. Although they do not exhibit shape memory or superelasticity, stents made from these materials also must undergo significant strain deformations in use.

FIG. 1 illustrates one of many stent designs that are used to facilitate this compression and expansion. This design uses ring shaped “struts” 12, each one having corrugations that allow it to be collapsed to a small diameter. Bridges 14, a.k.a. nodes, that also must flex in use connect the struts. Many other types of expandable geometries, such as helical spirals, braided and woven designs and coils, are known in the field and are used for various purposes.

One disadvantage of stents made from nitinol and many other alloys is that the metals used often have low atomic numbers and are, therefore, relatively poor X-ray absorbers. Consequently, stents of typical dimensions are difficult or impossible to see with X-rays when they are being manipulated or are in place. Such devices are called radio transparent. There are many advantages that would result from being able to see a stent in an X-ray. For example, radiopacity, as it is called, would result in the ability to precisely position the stent initially and in being able to identify changes in shape once it is in place that may reflect important medical conditions.

Many methods are described in the prior art for rendering stents or portions of stents radiopaque. These include filling cavities on the stent with radiopaque material (U.S. Pat. No. 6,635,082; U.S. Pat. No. 6,641,607), radiopaque markers attached to the stent (U.S. Pat. No. 6,293,966; U.S. Pat. No. 6,312,456; U.S. Pat. No. 6,334,871; U.S. Pat. No. 6,361,557; U.S. Pat. No. 6,402,777; U.S. Pat. No. 6,497,671; U.S. Pat. No. 6,503,271; U.S. Pat. No. 6,554,854), stents comprised of multiple layers of materials with different radiopacities (U.S. Pat. No. 6,638,301; U.S. Pat. No. 6,620,192), stents that incorporate radiopaque structural elements (U.S. Pat. No. 6,464,723; U.S. Pat. No. 6,471,721; U.S. Pat. No. 6,540,774; U.S. Pat. No. 6,585,757; U.S. Pat. No. 6,652,579), coatings of radiopaque particles in binders (U.S. Pat. No. 6,355,058), and methods for spray coating radiopaque material on stents (U.S. Pat. No. 6,616,765).

All of the prior art methods for imparting radiopacity to stents significantly increase the manufacturing cost and complexity and/or render only a small part of the stents radiopaque. The most efficient method would be to simply apply a conformal coating of a fully dense radiopaque material to all surfaces of the stent. The coating would have to be thick enough to provide good X-ray contrast, biomedically compatible and corrosion resistant. More challenging, however, it would have to be able to withstand the extreme strains in use without cracking or flaking and would have to be ductile enough that the important thermomechanical properties of the stent are preserved. In addition, the coatings must withstand the constant flexing of the stent that takes place because of the expansion and contraction of blood vessels as the heart pumps.

Physical vapor deposition techniques, such as sputtering, thermal evaporation and cathodic arc deposition, can produce dense and conformal coatings of radiopaque materials like gold, platinum, tantalum, tungsten and others. Physical vapor deposition is widely used and reliable. However, coatings produced by these methods do not typically adhere well to substrates that undergo strains of up to 8% as required in this application. This problem is recognized in U.S. Pat. No. 6,174,329, which describes the need for protective coatings over radiopaque coatings to prevent the radiopaque coatings from flaking off when the stent is being used.

Another important limitation of radiopaque coatings deposited by physical vapor deposition is the temperature sensitivity of nitinol and other stent materials. As mentioned, shape memory biomedical devices are made with values of A_{f} close to but somewhat below normal body temperature. If nitinol is raised to too high a temperature for too long its A_{f} value will rise and sustained temperatures above 300-400 C will adversely affect typical A_{f} values used in stents. Likewise, if stainless steel is raised to too high a temperature, it can lose its temper. Other stent materials would also be adversely affected. Therefore, the time-temperature history of a stent during the coating operation is critical. In the prior art it is customary to directly control the temperature of a substrate in such a situation, particularly one with a very low thermal mass such as a stent. This is usually accomplished by placing the substrate in thermal contact with a large mass, or heat sink, whose temperature is controlled. This process is known as controlling the temperature directly or direct control. Because of its shape and structure, controlling the temperature of a stent directly during coating would be a challenging task. Moreover, the portion of the stent in contact with the heat sink would receive no coating and the resulting radiographic image could be difficult to interpret.

Accordingly, there is a need in the art for biomedical devices having radiopaque coatings thick enough to provide good x-ray contrast, biomedical compatibility and corrosion resistance. Further, the coating needs to withstand the extreme strains in use without cracking or flaking and be sufficiently ductile so that the thermo-mechanical properties of the device are preserved.

Another serious disadvantage of stents and other medical devices made from biocompatible alloys discussed above, is that such alloys do not have surfaces capable of holding or retaining a drug or other desirable composition or material due to generally smooth non-porous surfaces of biocompatible metals.

### SUMMARY

The present invention is directed towards a biocompatible medical device having a biocompatible outer coating thereon which outer coating is able to withstand the strains produced in the use of the device without delamination. The outer coating may have a number of purposes such as being radiopaque to provide better imaging, or being somewhat porous to permit the incorporation of a medicinal drug or other compound or both.

When the outer coating is to permit better imaging, it is usually formed from a biologically inert heavy metal such as gold, platinum, or tantalum. When the coating is for other purposes it may be made of lighter metals and their alloys such as titanium or vanadium or inert alloys such as alloys of iron with chromium and/or nickel.

When the present invention is directed towards a medical device having a radiopaque outer coating, it is able to withstand the strains produced in the use of the device without delamination.

A medical device in accordance with the present invention can include a body at least partially comprising a nickel and titanium alloy or some other suitable material and a Ta coating on at least a portion of the body; wherein the Ta coating is sufficiently thick so that the device is radiopaque and the Ta coating is able to withstand the strains produced in the use of the device without delamination. The Ta coating can consist of either the bcc crystalline phase or the tetragonal crystalline phase. The coating thickness is preferably between approximately 3 and 10 microns. The device can be a stent or a guidewire, for example. The coating preferably is porous. The coating is applied via one of a generally oblique coating flux or a low energy coating flux.

A process for depositing a Ta or other metal layer on a medical device consisting of the steps of: maintaining a background pressure of inert gas in a sputter coating system containing a Ta (or other metal) sputter target; applying a voltage to the target to cause sputtering; and sputtering for a period of time to produce the desired coating thickness. When the metal is tantalum, the Ta layer preferably has an emissivity in the visible spectrum of at least 80%. The device preferably is not directly heated or cooled and the equilibrium temperature of the device during deposition is controlled indirectly by the process. The equilibrium temperature preferably is between 150° and 450° C. A voltage, ac or dc, can be applied steadily or in pulses to the medical device during the process. An initial high voltage, preferably between 100 and 500 volts, can be applied to preclean the device for a first period of time, preferably between 1 minute and 20 minutes. A second, lower voltage, preferably between 50 and 200 volts, can be applied for a period of time, preferably between 1 and 3 hours. Preferably, the inert gas is from the group comprising Ar, Kr and Xe. Preferably, the voltage on the target(s) produces a deposition rate of 1 to 4 microns per hour. The target preferably is a cylinder or a plate.

A medical device comprises a body having an outer layer and a radiopaque and/or porous coating on at least a portion of the outer layer; wherein the coating is applied using a physical vapor deposition technique.

### BRIEF DESCRIPTION OF THE DRAWINGS

These and other features, aspects and advantages of the present invention will become better understood with regard to the following description, appended claims, and accompanying drawings where:

FIG. 1 illustrates a stent found in the prior art;

FIG. 2 is a top view of a Ta target surrounding stents;

FIG. 3 is a side cross-sectional view of the target surrounding stents of FIG. 2;

FIG. 4 illustrates a cross section of a conformal coating of Ta on a strut 12 of a stent;

FIG. 5 is a graph showing the reflectance of a Ta coating made according to the present invention with respect to wavelength;

FIG. 6 is a graph showing the x-ray diffraction pattern of a Ta coating made according to the present invention;

FIG. 7 is a side cross-sectional view of the target surrounding stents in position C of FIG. 3 with a plate above the stents;

FIG. 8 is a top view of a Ta target surrounding stents;

FIG. 9 is a side cross-sectional view of the target surrounding stents of FIG. 8;

FIG. 10 is a side elevation view of stents positioned beside a planar target at a high angle of incidence;

FIG. 11 shows a scanning electron micrograph of the surface of a Ta coating applied to a polished stainless steel surface;

FIG. 12 shows an atomic force microscopy image of a Ta coating made according to another preferred embodiment of the present invention and applied to a polished nitinol substrate; and

FIG. 13 shows an X-ray diffraction pattern of a coating made according to another preferred embodiment of the present invention.

### DESCRIPTION

The present invention is directed towards a medical device having a metallic outer coating that is able to withstand the strains produced in the use of the device without delamination. The coating may commonly be a radiopaque metal coating, e.g. of gold, platinum, tantalum or alloys of such metals with other metals.

Tantalum has a high atomic number and is also biomedically inert and corrosion resistant, making it an attractive material for radiopaque coatings in this application, although other materials may be used, such as, but not limited to, platinum, gold or tungsten. It is known that 3 to 10 microns of Ta is sufficiently thick to produce good X-ray contrast. However, because Ta has a melting point of almost 3000 C, any coating process must take place at a low homologous temperature (the ratio of the deposition temperature to the melting temperature of the coating material in degrees Kelvin) to preserve the A_{f} values of the stents as described previously. It is well known in the art of physical vapor deposition that low homologous coating temperatures often result in poor coating properties. Nevertheless, we have unexpectedly found that radiopaque Ta coatings deposited under the correct conditions are able to withstand the strains inherent in stent use without unacceptable flaking.

Still more remarkable is the fact that we can deposit these adherent coatings at high rates with no direct control of the stent temperature without substantially affecting A_{f}. Since normal body temperature is 37 C, the A_{f} value after coating should be less than this temperature to avoid harming the thermomechanical properties of the nitinol. The lower A_{f} is after coating the more desirable the process.

For a thermally isolated substrate, the equilibrium temperature will be determined by factors such as the heat of condensation of the coating material, the energy of the atoms impinging on the substrate, the coating rate, the radiative cooling to the surrounding chamber and the thermal mass of the substrate. It is surprising that this energy balance permits high-rate coating of a temperature sensitive low mass object such as a stent without raising the temperature beyond acceptable limits. Eliminating the need to directly control the temperature of the stents significantly simplifies the coating operation and is a particularly important consideration for a manufacturing process.

This patent relates to coatings that render biomedical devices including intraluminal biomedical devices radiopaque and that withstand the extremely high strains inherent in the use of such devices without unacceptable delamination. Specifically, it relates to coatings of Ta having these properties and methods for applying them that do not adversely affect the thermomechanical properties of stents.

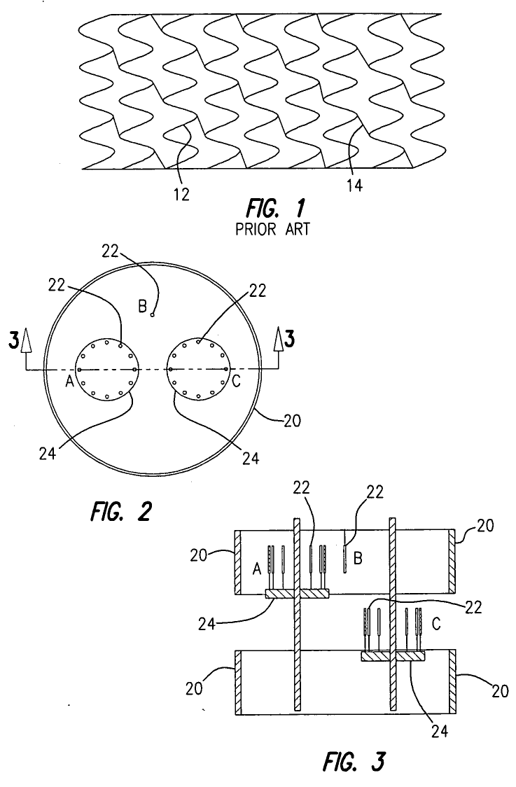

An unbalanced cylindrical magnetron sputtering system described in U.S. Pat. No. 6,497,803 B2, which is incorporated herein by reference, was used to deposit the coatings. FIGS. 2 and 3 illustrate the setup. Two Ta targets 20, each 34 cm in diameter and 10 cm high, separated by 10 cm, were used. They were driven with either DC power or AC power at 40 kHz. Xenon or krypton was used as the sputter gas. The total power to both cathodes was either 2 kW or 4 kW and a bias of either −50 V or −150 V was applied to the stents during coating. Other devices well known to those in the art, such as vacuum pumps, power supplies, gas flow meters, pressure measuring equipment and the like, are omitted from FIGS. 2 and 3 for clarity.

In each coating run, stents 22 were placed at one of three positions, as shown in FIGS. 2 and 3:

Position A—The stents were held on a 10 cm diameter fixture 24 that rotated about a vertical axis, which was approximately 7 cm from the cathode centerline. The vertical position of the stents was in the center of the upper cathode. Finally, each stent was periodically rotated about its own vertical axis by a small “kicker”, in a manner well known in the art.

Position B—The stents 22 were supported from a rotating axis that was approximately 7 cm from the chamber centerline. The vertical position of the stents was in the center of the upper cathode.

Position C—The stents 22 were on a 10 cm diameter fixture or plate 24 that rotated about a vertical axis, which was approximately 7 cm from the cathode centerline. The vertical position of the stents was in the center of the chamber, midway between the upper and lower cathodes. Finally, each stent was periodically rotated about its own vertical axis with a “kicker.”

Prior to coating, the stents were cleaned with a warm aqueous cleaner in an ultrasonic bath. Crest 270 Cleaner (Crest Ultrasonics, Inc.) diluted to 0.5 pounds per gallon of water was used at a temperature of 55 C. This ultrasonic detergent cleaning was done for 10 minutes. The stents were then rinsed for 2 minutes in ultrasonically agitated tap water and 2 minutes in ultrasonically agitated de-ionized water. The stents were then blown dry with nitrogen and further dried with hot air. The manner in which the stents were cleaned was found to be very important. When the stents were cleaned ultrasonically in acetone and isopropyl alcohol, a residue could be seen on the stents that resulted in poor adhesion. This residue may be a consequence of material left after the electropolishing process, which is often done using aqueous solutions.

The Ta sputtering targets were preconditioned at the power and pressure to be used in that particular coating run for 10 minutes. During this step a shutter isolated the stents from the targets. This preheating allowed the stents to further degas and approach the actual temperature of the coating step. After opening the shutter, the coating time was adjusted so that a coating thickness of approximately 10 microns resulted. At a power of 4 kW the time was 2 hours and 15 minutes and at a power of 2 kW the time was 4 hours and 30 minutes. These are very acceptable coating rates for a manufacturing process. The stents were not heated or cooled directly in any way during deposition. Their time-temperature history was determined entirely by the coating process.

FIG. 4 illustrates the cross section of a conformal coating of Ta 40 on a strut 12, shown approximately to scale for a 10-micron thick coating. Stents coated in this manner were evaluated in several ways. First, they were pressed into adhesive tape to see if there was any flaking or removal when the tape was peeled away. Next, the stents were flexed to their maximum extent and examined for flaking. In all cases this flexing was done at least three times and in some cases it was done as many as ten times. Finally, the A_{f} values for the stents were measured by determining the temperature at which they recovered their original shape using a water bath.

Table 1 summarizes the results. The level of flaking and A_{f} temperatures at positions A and B were very similar in the experiments and were averaged to produce the values shown. The level of flaking was ranked using the following procedure:
Level 5: Approximately 10% or more of the coated area flaked.
Level 4: Between approximately 5% and 10% of the coated area flaked.
Level 3: Between approximately 1% and 5% of the coated area flaked.
Level 2: Between approximately 0.1% and 1% of the coated area flaked.
Level 1: An occasional flake was observed, but less than approximately 0.1% of the coated area flaked.
Level 0: No flakes were observed.

Depending on the application, some level of flaking may be tolerated and we consider Level 2, Level 1 or Level 0 flaking acceptable.

TABLE 1

| Run No | Power | Gas | Bias | AC/DC | Flaking | Af | Appearance |
|---|---|---|---|---|---|---|---|
| 1 | 2 kW | Xe | 50 | AC | 5 | 29 | Dull mottled |
| | | | | | | | appearance |
| 2 | 2 kW | Kr | 150 | AC | 0 | 59 | Shiny metallic |
| | | | | | | | appearance |
| 3 | 4 kW | Kr | 50 | AC | 4 | 57 | Dull mottled |
| | | | | | | | appearance |
| 4 | 4 kW | Xe | 150 | AC | 0 | 60 | Shiny metallic |
| | | | | | | | appearance |
| 5 | 2 kW | Kr | 50 | DC | 0 | 23 | Black |
| | | | | | | | appearance |
| 6 | 2 kW | Xe | 150 | DC | 0 | 27 | Dull mottled |
| | | | | | | | appearance |
| 7 | 4 kW | Xe | 50 | DC | 4 | 32 | Shiny metallic |
| | | | | | | | appearance |
| 8 | 4 kW | Kr | 150 | DC | 1 | 38 | Shiny metallic |
| | | | | | | | appearance |

It can be seen from the results with respect to positions A and B that a major factor in determining adhesion is the bias voltage. A bias of −150 V produces much better adhesion overall than a bias of −50 V. This is consistent with many reports in the literature that higher substrate bias produces better adhesion in many applications. However, it also produces greater heating at a given power, as determined by the A_{f} values.

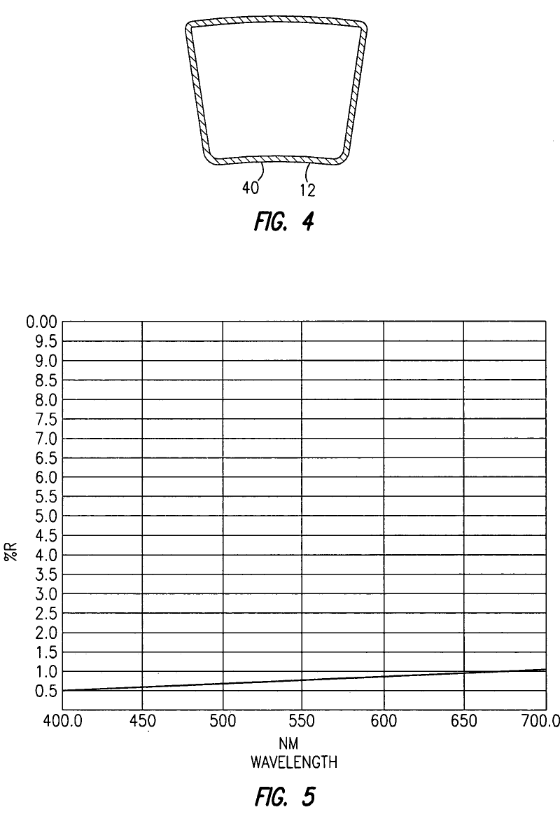

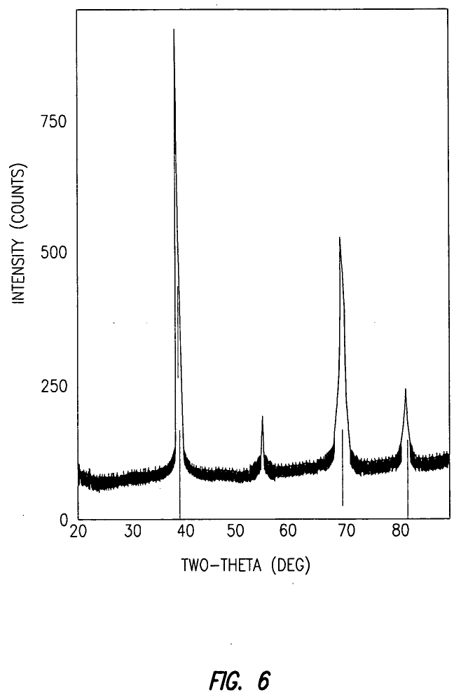

An important exception to the need for high bias to produce good adhesion is Run Number 5, which has both excellent adhesion and the lowest value for A_{f} among the coatings. Moreover, the coating appearance of Run Number 5 was black, which could be appealing visually. This is indicative of a very high emissivity in the visible spectrum, characteristic of a so-called black body. As charted in FIG. 5, the reflectance was measured to be about 0.5% at a wavelength of 400 nm and rises to about 1.10% at 700 nm. Because this substrate does not transmit significant radiation, we can use the relationship that r+a=1, where r is the reflectance and a is the absorptance of the coating. Therefore, the absorptance is approximately 99% in this case. Because absorptance and emittance are the same (see for example “University Physics,” third edition by Sears and Zamansky (Addison Wesley 1964, pp. 376-378)), this is an emissivity of approximately 99% or greater across the visible spectrum.

The combination of a very low A_{f} and excellent adhesion is very surprising. Without being bound to this explanation, one possibility consistent with the observed results is that the coating is very porous. Low homologous temperatures (the ratio of the substrate temperature during coating to the melting point of the coating material, in degrees Kelvin) are known to produce open, columnar coating structures. Those skilled in the art will recognize that the porous coatings we are describing are those sometimes called Zone 1 coatings for sputtered and evaporated materials (see, for example, ‘High Rate Thick Film Growth” by John Thornton, Ann. Rev. Mater. Sci., 1977, 239-260).

The observed black appearance may be the result of an extremely porous coating. It is also known in the art that such morphology is also associated with very low coating stress, since the coating has less than full density. However, even if this explanation is correct, the excellent adhesion is very surprising. Typically the coating conditions that lead to such porous coatings result in very poor adhesion and we were able to aggressively flex the coating with no indication of flaking.

Another possible consequence of the high emissivity of the coating is the fact that the radiative cooling of the stent during coating is more effective than that of a low emissivity, shiny surface, thereby helping to maintain a low coating temperature.

Furthermore, as described in Utility patent application Ser. No. 11/040,433, which is incorporated herein by reference, sputtered Ta typically exists in one of two crystalline phases, either tetragonal (known as the beta phase) or body centered cubic (known as the alpha phase). The alpha phase of Ta is much more ductile than the beta phase and can withstand greater strains. Therefore, the alpha phase of Ta may be more desirable in this application. FIG. 6 is an X-ray diffraction pattern of a coating made under the conditions of Run No. 5 described above, showing that the coating is alpha tantalum. It is known in the art that sputtering Ta in Kr or Xe with substrate bias can result in the alpha phase being deposited. See, for example, Surface and Coatings Technology 146-147 (2001) pages 344-350. However, there is nothing in the prior art or in our experience to suggest that alpha Ta coatings of 10 microns thickness can withstand the very high strains inherent in the use of stents without delamination and coating failure. There is also nothing in the prior art to suggest that alpha Ta can be deposited in such an open, porous structure.

An open, porous structure may have other advantages as well. For example, the microvoids in the coating would permit the incorporation of drugs or other materials that diffuse out over time. In the art, drug-eluting coatings on stents are presently made using polymeric materials. A porous inorganic coating would allow drug-eluting stents to be made without polymeric overcoats.

Surprisingly, the stents at position C as shown in FIG. 3 all had adhesion equal to or better than the stents at positions A and B, regardless of conditions. Table 2 illustrates the surprising results. (NA indicates coating runs for which no data was taken at those positions.) The stents at position C always had very little or no flaking, even under coating conditions where stents in positions A or B had significant flaking. As can be seen from Table 2, this is true over a wide range of coating conditions. The A_{f} values of the stents in position C were comparable to those in the other positions, and in the case of the AC coatings they were sometimes significantly lower. Stents in the C position that were sputter coated in Kr at a pressure of 3.4 mTorr, an AC power of 2 kW with −50 V bias (Run Nos. 2 and 3) had a metallic appearance and an A_{f} between 38 and 42 C. Those coated in the C position using Kr at a pressure of 3.4 mTorr, a DC power of 2 kW and −50 V bias (Run No. 8) were black in appearance with an A_{f} of only 24 C. An A_{f} of 24 C is virtually unchanged from the A_{f} values before coating. Both the metallic and the black samples had excellent adhesion. The fact that position C is preferable for adhesion and A_{f} in virtually every case is unexpected.

TABLE 2

| Total | | | | | | |
|---|---|---|---|---|---|---|
| Power | Gas | Bias | AC/DC | Position A | Position B | Position C |
| 2 kW | Xe | 50 | AC | Af = 29 C | Af = 28 C | Af = 30 C |
| | | | | 5 | 5 | 0 |
| 2 kW | Kr | 150 | AC | Af = 59 C | NA | Af = 42 C |
| | | | | 0 | | 0 |
| 2 kW | Kr | 150 | AC | Af = 52 C | Af = 45 C | Af = 38 C |
| | | | | 0 | 0 | 0 |
| 4 kW | Kr | 50 | AC | Af = 56 C | Af = 58 C | NA |
| | | | | 4 | 4 | |
| 4 kW | Kr | 150 | AC | Af > 55 C | Af > 55 C | NA |
| | | | | 0 | 0 | |
| 4 kW | Kr | 150 | AC | NA | Af > 55 C | NA |
| | | | | | 0 | |
| 4 kW | Xe | 150 | AC | NA | Af > 55 C | NA |
| | | | | | 0 | |
| 2 kW | Kr | 50 | DC | Af = 25 C | Af = 22 C | Af = 24 C |
| | | | | 0 | 0 | 0 |
| 4 kW | Xe | 150 | DC | Af = 37 C | Af = 37 C | Af = 38 C |
| | | | | 1 | 5 | 0 |
| 4 kW | Xe | 50 | DC | Af = 32 C | Af = 33 C | Af = 31 C |
| | | | | 3 | 5 | 1 |
| 4 kW | Kr | 150 | DC | Af = 38 C | Af = 38 C | Af = 49 C |
| | | | | 1 | 0 | 0 |
| 2 kW | Xe | 150 | DC | Af = 25 C | Af = 29 C | Af = 25 C |
| | | | | 0 | 0 | 1 |

Stents in position C receive a generally more oblique and lower energy coating flux than stents in positions A or B. By an oblique coating flux we mean that the majority of the depositing atoms arrive in directions that are not generally perpendicular to the surface being coated. Some of the atoms arriving at the surfaces of the stents in position C from the upper and lower targets will have done so without losing significant energy or directionality because of collisions with the background sputter gas. Those atoms, most of which will come from portions of the targets close to the stents as seen in FIGS. 2 and 3, will create an oblique coating flux. Other atoms will undergo several collisions with the background gas and lose energy and directionality before arriving at the substrate surfaces. Those atoms, which will generally come from portions of the targets at greater distances, will form a low energy coating flux.

Westwood has calculated (“Calculation of deposition rates in diode sputtering systems,” W. D. Westwood, Journal of Vacuum Science and Technology, Vol. 15 page 1 (1978)) that the average distance a Ta atom goes in Ar at 3.4 mTorr before its energy is reduced to that of the background gas is between about 15 and 30 cm. (The distance would be somewhat less in Kr and the exact value depends on the initial energy of the Ta atom.) Because our cylindrical targets have an inside diameter of approximately 34 cm, substrates placed in the planes of the targets (positions A and B) receive a greater number of high energy, normal incidence atoms than those placed between the targets (position C).

The geometry of the cylindrical magnetron arrangement shown in FIGS. 2 and 3 assures that atoms arriving at the surface of the stents in position C will do so either at relatively oblique angles or with relatively low energy. Typically, sputtered atoms leave the target surface with average kinetic energies of several electron volts (eV). As described by Westwood, after several collisions with the background gas the sputtered atoms lose most of their kinetic energy. By low energy, we are referring to sputtered atoms that have average energies of approximately 1 eV or less. Westwood's calculations can be used to estimate the target to substrate spacing required to achieve this low average energy for a given sputtering pressure. Furthermore, it is well known to those skilled in the art that atoms deposited by evaporation have average energies below approximately one eV when they leave the evaporation source. Therefore, scattering from the gas in the chamber is not required to produce a low energy coating flux in the case of evaporated coatings.

In summary, referring to FIGS. 2 and 3, when the stents are close to the targets, where the arriving Ta atoms have lost little energy, the atoms arrive at oblique angles. And when the stents move closer to the center of the chamber where the arrival angles are less oblique, they are farther from the target surface so that the arriving Ta atoms have lost energy through gas collisions.

It is widely known in the art that when the atoms in a PVD process arrive with low energies or at oblique angles to the substrate surface, the result is a coating that is less dense than a coating made up of atoms arriving at generally normal incidence or with higher energies. The black appearance of the low power DC coatings deposited with low substrate bias (Run 5 in Table 1 and Run 8 in Table 2) may be the result of considerable coating porosity. Normally low-density PVD coatings are not desirable, but we have found that conditions that result in relatively low density or porous coatings produce very desirable results in this application.

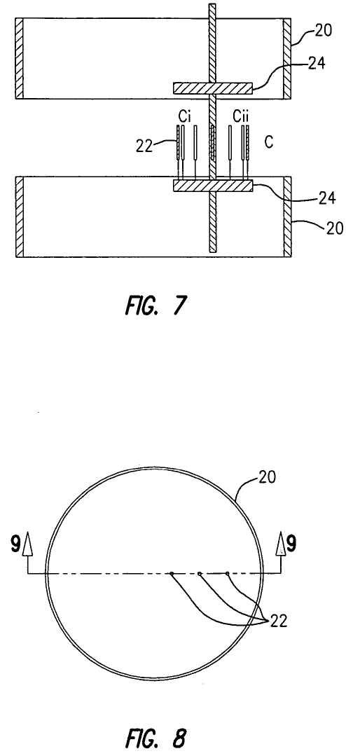

Further evidence of the importance of the coating geometry is seen in the following experiment. A number of coatings were done in Kr at a pressure of 3.4 mTorr, a DC power of 2 kW and a bias of −50 V using the fixture shown in FIGS. 2 and 3 in position C. As before, the stents were rotating about the vertical rod as well as about their own vertical axes. The coated stents made this way were matte black at the bottom but had a slightly shinier appearance at the top. In contrast, when coatings were done on stents 22 under identical conditions, except that a second plate 24 was placed above the stents as shown in FIG. 7, the stents were a uniform black from bottom to top.

The non-uniformity in appearance that resulted with the fixturing shown in FIGS. 2 and 3 in position C indicates that the coating structure depends on the details of how the stents and sputter targets are positioned relative to one another. As discussed earlier, when the stents are in position Ci in FIG. 7, they receive very oblique incidence material from portions of the targets that are close, while the coating material that arrives from other portions of the target has to travel farther. Therefore, all of the coating flux has arrived at high angles or has traveled a considerable distance and has lost energy and directionality through collisions with the sputtering gas. When the stents are in position Cii in FIG. 7, however, they receive a somewhat less oblique coating from all directions. In the configuration shown in FIG. 3, position C the bottoms of the stents are shielded from the more direct flux from the bottom target by the plate that holds them, but the tops of the stents are not similarly shielded from the more direct flux coming from the top target. By adding the plate above the stents shown in FIG. 7, the more direct coating flux is shielded at all points on the stents and the coating material either arrives at relatively oblique incidence or after scattering from the background gas and losing energy and directionality. The plate above the stents restores the symmetry of the situation and the coatings on the stents become uniformly black overall.

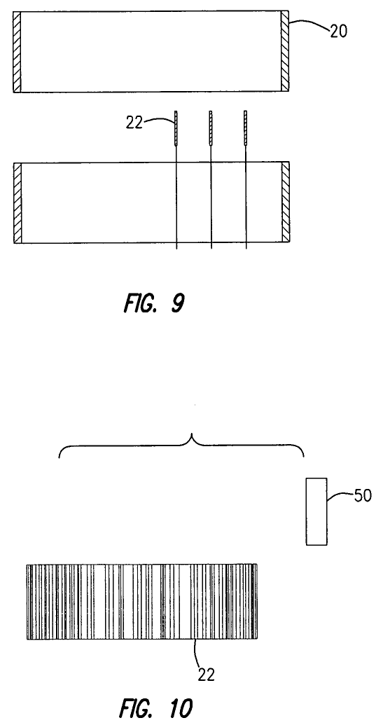

Other methods of positioning and moving the substrates within the chamber can also produce results similar to those described above and are within the scope of the invention. In another experiment three stents were located as shown in FIGS. 8 and 9. All three stents 22 were held fixed at their positions within the chamber and were rotated about their individual vertical axes during the coating run. The innermost stent was 3 cm from the cathode centerline, the middle stent was 7 cm from the cathode centerline and the outermost stent was 11 cm from the cathode centerline. The deposition was done at a DC power of 2 kW, a Kr pressure of 3.4 mTorr and with the stents biased at −50 V. These are the same conditions used in Run No. 8 in Table 2. All three stents had a matte black appearance and exhibited excellent adhesion when tested. Therefore, stents placed at virtually any radial position within the cathodes and rotating about their individual vertical axes will receive a satisfactory coating, provided they are located between the targets in the axial direction.

An alternative, although less desirable, approach to oblique incidence coatings or large target to substrate distances in order to reduce the energy of the arriving atoms through collisions is to raise the pressure of the sputtering gas.

Sputtering takes place under conditions of continuous gas flow. That is, the sputtering gas is brought into the chamber at a constant rate and is removed from the chamber at the same rate, resulting in a fixed pressure and continuous purging of the gas in the chamber. This flow is needed to remove unwanted gases, such as water vapor, that evolve from the system during coating. These unwanted gases can become incorporated in the growing coating and affect its properties.

The high vacuum pumps used in sputtering, such as diffusion pumps, turbomolecular pumps and cryogenic pumps, are limited with respect to the pressure that they can tolerate at their openings. Therefore, it is well known that in order to achieve high sputtering pressures it is necessary to “throttle” such pumps, or place a restriction in the pump opening that permits the chamber pressure to be significantly higher than the pressure at the pump. Such “throttling” necessarily reduces the flow of gas through the chamber, or gas throughput. Surprisingly, we have found that the adherence of the coatings is improved at high gas throughputs.

In one experiment, a cylindrical magnetron cathode with an inside diameter of 19 cm and length of 10 cm was used to coat a stent with Ta at a sputtering pressure of 30 mTorr in Ar. In order to achieve this pressure, it was necessary to throttle the turbomolecular high vacuum pump on the vacuum system. The Ar flow during this coating was 0.63 Torr-liters per second, corresponding to a throttled pumping speed of 21 liters per second. The stent was placed in the center of the cathode, approximately 9 cm from the target surface. The sputtering power to the cathode was 200 W. According to Westwood's calculations, the average distance a Ta atom travels in Ar at 30 mTorr before reaching thermal velocities is between 1.7 and 3.4 cm, depending on its initial energy. Therefore, these coating conditions should result in a very low-density coating. The black appearance of the coated stent confirmed that this was the case. However, the coating had very poor adhesion.

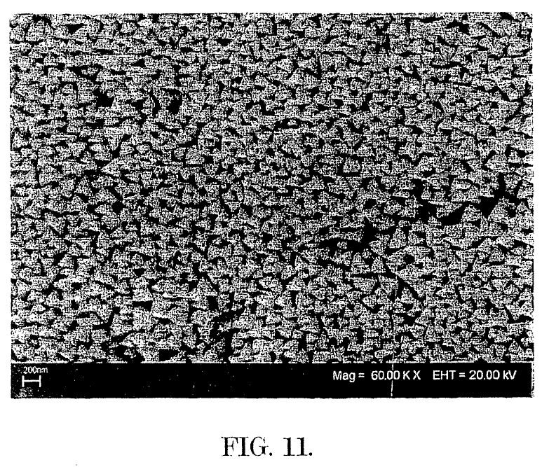

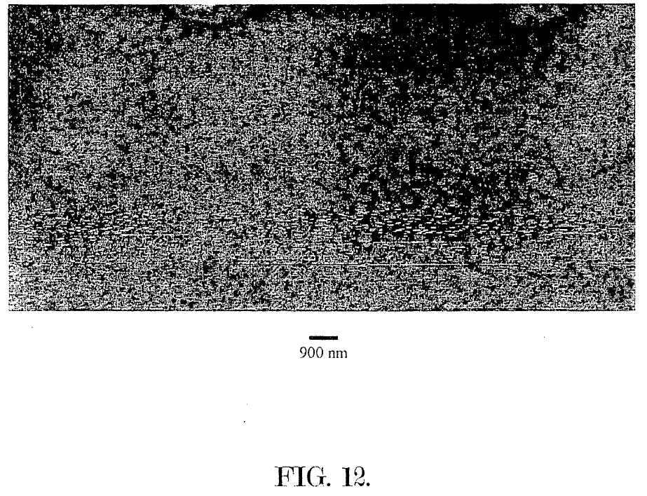

In another experiment, coatings were done on stents in the C position using the 34 cm diameter dual cathode shown in FIGS. 2 and 3. The sputtering gas was Kr at a pressure of 3.4 mTorr. A DC power of 2 kW was used together with a substrate bias of −50 V, the conditions of Run No. 8 in Table 2. The Kr flow was 28 standard cubic centimeters per minute, or 0.36 Torr-liters per second. At a pressure of 3.4 mTorr, this corresponds to a throttled pumping speed of 104 liters per second during the process. The resulting black coatings all flaked at levels between level 1 and level 3 when tested. The position of the pump throttle was then changed and the Kr flow was increased to 200 standard cubic centimeters per minute or 2.53 Torr-liters per second. Coatings were done on stents in the C position at the same power, pressure and bias levels as before. The only difference was that the throttled pumping speed during this process was 744 liters per second. In this case there were no flakes or cracks in the coating evident after testing. A scanning electron micrograph of the surface of a coating applied to a polished stainless steel surface under these conditions is shown in FIG. 11. The open, porous nature of the coating is clearly visible.

Based on the foregoing results, we conclude that adequate adhesion does not result at low gas throughputs, which are usually necessary to achieve high sputtering pressures. The sputtering pressure and system geometry must be chosen together so that the coating flux arrives at the substrate surface either at high angles of incidence or after the sputtered atoms have traveled a sufficient distance from the target to reduce their energies significantly.

While the geometry of a cylindrical magnetron makes this possible in an efficient way, as we have shown, the same results can be accomplished using planar targets as well. In the case of planar targets, the requirement is to place the substrates far enough from the target surface(s) that a large target-to-substrate distance is achieved. Alternatively, the substrates could be placed to the side of a planar target so that the material arrives at high incidence angles. This configuration is illustrated in FIG. 10. Of course, the stent positions 22 shown in the case of planar target 50 make inefficient use of the coating material. Nevertheless, FIG. 10 illustrates how the inventive method could be used with geometries other than cylindrical magnetrons.

We have also discovered that the initial coating conditions can influence the microstructure and crystalline phase of our coatings while preserving excellent adhesion. In one experiment, stents were loaded in Position C using the setup shown in FIG. 7 with 34 cm diameter targets. With the shutter closed, the two Ta targets were operated at 2 kW (1 kW each) at a Kr pressure of 3.6 mT and a Kr flow of 200 standard cubic centimeters per minute. After five minutes, and with the shutter still closed, a voltage of −200 V was applied to the stents in order to plasma clean them. The shutter was opened after five additional minutes and the coating was begun with a −200 V bias still applied to the stents. These conditions were maintained for two minutes, at which time the voltage on the stents was reduced to −50 V and the coating was deposited under these conditions for 180 minutes. There was no flaking evident on these stents.

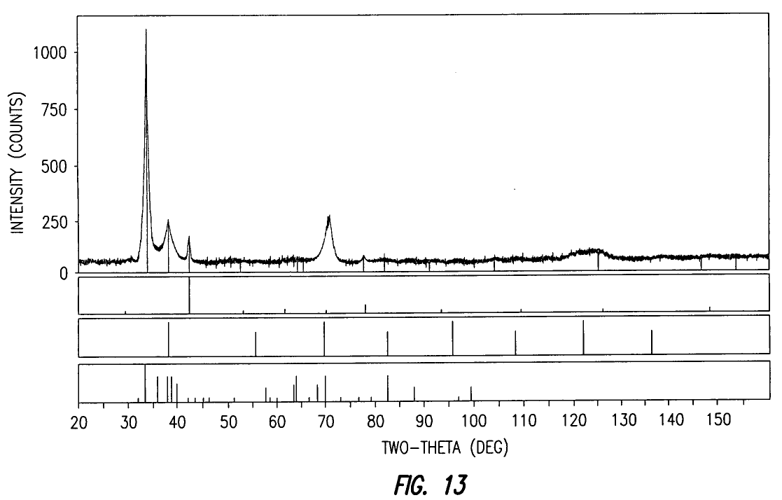

Except for the initial five minutes of plasma cleaning and two minutes of −200 V bias sputtering, the conditions in the example above were the same as those that produced the structure shown in FIG. 11 and the bcc crystalline phase. FIG. 12 is an atomic force microscope image of the resulting coating showing that the microstructure is changed by the initial conditions. While the features in FIGS. 11 and 12 are similar and both are porous coatings, a close analysis shows that the structures in FIG. 11 are approximately 100 to 200 nm in size, while those in FIG. 12 are about twice as large. Moreover, the X-ray diffraction pattern in FIG. 13 shows that the crystalline phase of this coating shown in FIG. 12 was primarily tetragonal, with some bcc present. The reflectance of this coating went from approximately 11% at a wavelength of 400 nm to approximately 17% at a wavelength of 700 nm.

Without being bound to this explanation, we are led to believe that a very important factor in the excellent adhesion of our coatings is the porous structure, which is promoted by oblique incidence and/or low energy deposition.

Although the present invention has been described in considerable detail with reference to certain preferred versions thereof, other versions are possible. For example, a device other than a stent can be coated with Ti or another inert metal or another material, e.g. to form a porous coating that will retain and slowly release a drug or other material or that will modify the coating to provide another benefit, e.g. increased biocompatibility, corrosion resistance or bioincorportation, e.g. permitting the growth of tissue into the coating or bio resistance, preventing the growth of tissue into the coating. Examples of such materials are slow release antibiotics, inert polymers, and slow release anticoagulants, The pores in the coating are of such a size that the drug (medicament) is retained over an extended time period and slowly released, e.g. over a time period of from a week to several years. Therefore, the spirit and scope of the appended claims should not be limited to the description of the preferred versions contained herein.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function should not be interpreted as a “means” or “step” clause as specified in 35 U.S.C. §112.

We claim:

1. A medical device comprising:
a. a body at least partially comprising a radio transparent material; and
b. a porous Ta coating on at least a portion of the body; wherein the Ta coating is sufficiently thick so that the device is radiopaque and the Ta coating is able to withstand the strains produced in the use of the device without unacceptable flaking; wherein the Ta is applied to the body via one of a generally oblique coating flux or a low energy coating flux.

2. The medical device of claim 1 in which said Ta coating consists primarily of the bcc crystalline phase.

3. The medical device of claim 1 in which said coating thickness is between approximately 3 and 10 microns.

4. The medical device of claim 1 in which said device is a stent.

5. The medical device of claim 1 in which said device is a guidewire.

6. The medical device of claim 1 wherein the device is an intraluminal device.

7. The medical device of claim 1 wherein the Ta coating is applied to the body by a physical vapor deposition process.

8. The medical device of claim 7 wherein the physical vapor deposition process includes one of the group of sputtering, cathodic arc deposition or thermal evaporation.

9. The medical device of claim 1 further comprising a material in the Ta coating, wherein the material is intended to diffuse out over time.

10. A process for depositing a Ta layer on a medical device consisting of the steps of:
a. maintaining a background pressure of inert gas in a sputter coating system containing at least one Ta sputter target;
b. applying a voltage to said Ta target to cause sputtering; and
c. sputtering for a period of time to produce the desired coating thickness;
wherein the Ta layer has an emissivity in the visible spectrum of at least 80% and wherein the Ta is applied to the medical device via one of a generally oblique coating flux or a low energy coating flux.

11. The process of claim 10 wherein the equilibrium temperature of said device during deposition is controlled indirectly by said process.

12. The process of claim 10 in which the equilibrium temperature is between 150 and 450 C.

13. The process of claim 10 in which a voltage is applied to said medical device during said process.

14. The process of claim 13 in which said voltage comprises an initial high voltage to preclean said device for a first period of time.

15. The process of claim 14 in which said initial high voltage is between 100 and 500 volts.

16. The process of claim 14 in which said first period of time is between 1 minute and 20 minutes.

17. The process of claim 13 in which said voltage comprises a second, lower voltage applied for a second period of time.

18. The process of claim 17 in which said lower voltage is between 10 and 100 volts.

19. The process of claim 17 in which said second period of time is between 1 hour and 5 hours.

20. The process of claim 10 in which said inert gas is from the group comprising Ar, Kr and Xe.

21. The process of claim 10 in which said voltage produces a deposition rate of 1 to 5 microns per hour.

22. The process of claim 10 wherein the Ta layer is porous.

23. The process of claim 22 further comprising the steps of incorporating a material into the pores, wherein the material is intended to diffuse out over time.

24. A medical device comprising:
a. a body having an outer layer; and
b. a radiopaque coating on at least a portion of the outer layer; wherein the coating is applied via one of a generally oblique coating flux or a low energy coating flux using a physical vapor deposition process.

25. A medical device comprising:
a. a body at least partially comprising a radio transparent material;
b. a Ta coating on at least a portion of the body; wherein the Ta coating is able to withstand the strains produced in the use of the device without unacceptable flaking and wherein the Ta is applied to the body via one of the generally oblique coating flux or a low energy coating flux.

26. A medical device comprising:
a. a body at least partially comprising a resilient biocompatible metallic material; and
b. a porous metallic coating on at least a portion of the body; wherein the metallic material is sufficiently adherent and flexible to prevent flaking from the body under conditions of use.

27. The device of claim 26 where the coating is sufficiently dense and thick so that the device is radiopaque wherein the metal coating is applied to the body via one of a generally oblique coating flux or a low energy coating flux.

28. The device of claim 26 where the coating is porous with sufficiently small pores to retain a medicament for a time period of from a week to several years.

29. The device of claim 28 wherein the medicament is an antibiotic.

30. A process for depositing a metallic layer on a medical device comprising the steps of:
a. maintaining a background pressure of inert gas in a sputter coating system containing at least one sputter target made of the metal of the intended metallic layer;
b. applying a voltage to said target to cause sputtering; and
c. sputtering for a period of time to produce the desired coating thickness; wherein the metal is applied to the medical device via one of a generally oblique coating flux or a low energy coating flux.
